(19) United States
(12) Patent Application Publication
(10) Pub. No.: US 2024/0392247 A1
(43) Pub. Date: Nov. 28, 2024

(54) BOVINE PLURIPOTENT STEM CELL-BASED MUSCLE ORGANOIDS, METHOD OF PREPARING THE SAME AND USES THEREOF

(71) Applicant: RAMOT AT TEL AVIV UNIVERSITY LTD., Tel Aviv (IL)

(72) Inventors: Gaya SAVYON, Tel Aviv (IL); Iftach NACHMAN, Tel Aviv (IL); Michelle HAUSER, Tel Aviv (IL); Lara AVNI, Tel Aviv (IL)

(21) Appl. No.: 18/791,781

(22) Filed: Aug. 1, 2024

Related U.S. Application Data
(63) Continuation of application No. PCT/IL2023/050162, filed on Feb. 16, 2023.
(60) Provisional application No. 63/325,203, filed on Mar. 30, 2022.

(51) Int. Cl. C12N 5/0775; A23L 13/00; C12N 5/00

(52) U.S. Cl. CPC C12N 5/0662; A23L 13/00; C12N 5/0018; C12N 2501/115; C12N 2501/155; C12N 2501/16; C12N 2501/41; C12N 2501/415; C12N 2506/02; C12N 2513/00; C12N 2527/00

(57) ABSTRACT

The present invention provides organoids comprising cultured bovine myogenic progenitor cells, methods for their preparation in suspension from bovine pluripotent stem cells, and uses thereof for preparing cultured meat.

### CROSS REFERENCE TO RELATED APPLICATIONS

This application is a Bypass Continuation of PCT/IL2023/050162 having International filing date of Feb. 16, 2023, which claims the benefit of priority of U.S. Provisional Patent Application No. 63/325,203, filed Mar. 30, 2022, the contents of which are incorporated herein by reference in their entirety.

### FIELD OF THE INVENTION

The present invention relates in general to the field of cultured meat. More specifically, the present invention relates to preparation of muscle tissue in culture from bovine embryonic stem cells.

### BACKGROUND OF THE INVENTION

The meat industry is one of the main causes of environmental degradation. Due to world population growth, meat consumption is expected to double between 1999 and 2050. One of the measures to counter the expected damage is an alternative solution to meat production called cultured meat (CM). CM is produced in-vitro using cell cultures. Since skeletal muscles are the main edible tissue used in most animal meat, generation of skeletal muscles in-vitro is required for the efficient production of CM. Although the generation of skeletal muscles in-vitro was achieved decades ago, existing protocols are not suitable for food applications due to two main reasons. First is the high cost of those protocols, which stems from expensive growth medium components, especially growth factors. Second is the inherent difficulty for scale up, as current bioreactor technologies support suspended cultures while most protocols for skeletal muscle generation in vitro are based on adherent 2D monolayer cultures.

Since skeletal muscles are the main edible tissue used in most animal meat, generation of skeletal muscles in large mass in-vitro is required for the efficient production of CM. One of the biggest challenges of the fast-developing cultivated meat industry is the large-scale production of muscle tissue at a reasonable cost. The proliferation rate, cost of medium, and complexity of the production process are all factors that affect the feasibility and cost of this process.

Jiwlawat et al. (Differentiation, 96: 70-81, 2017, “Differentiation and sarcomere formation in skeletal myocytes directly prepared from human induced pluripotent stem cells using a sphere-based culture”) discloses a protocol for the derivation of myogenic progenitor cells directly (without genetic modification) from human pluripotent cells.

Nachman et al. (iScience 25(1), 103556, 2022), is directed to emergence and patterning dynamics of mouse-definitive endoderm.

Patent Application, publication No. WO2020058732 is directed to polarized three-dimensional cellular aggregates (or gastruloids) generated in vitro from one or more pluripotent stem cells.

Budjhan et al. (eLife 11:e68925, 2022) discloses paraxial mesoderm organoids model development of human somites.

Nevertheless, there is an unmet need for an efficient, fast and cost-effective process for producing in-vitro bovine muscle cells, preferably within a tissue structure, while utilizing bovine somites generated from bovine embryonic stem cells.

### SUMMARY OF THE INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

Provided herein are myogenic progenitor cells derived from bovine embryonic stem cells (bESCs), methods of preparing the same in-vitro by culturing in suspension, and consequent uses thereof for the formation of bovine muscle cells or muscle tissue. In some embodiments, there are provided herein developmental path-based protocols for the generation, in culture, of bovine somites which can be further utilized for generation of bovine myogenic progenitor cells, muscle cells and muscle tissue.

According to some embodiments, there is provided a method for generating bovine myogenic progenitor cells, the method comprising: (a) culturing bovine pluripotent stem cells (bPSCs) in a first culture medium comprising at least one growth factor for a first time period until aggregates form and the cells express Brachyury, thereby obtaining mesodermal organoids; (b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and optionally somite-like structures; and (c) replacing the second culture medium with a third culture medium comprising at least one Wnt activator and at least one hedgehog inhibitor and incubating for a third time period until the cells express MyoD, thereby obtaining myogenic progenitor cells.

According to some embodiments, the bovine pluripotential stem cells are bovine embryonic stem cells (bESCs).

According to some embodiments, the at least one growth factor is selected from a fibroblast growth factor (FGF), an epidermal growth factor (EGF), a hepatocyte growth factor (HGF), an insulin-like growth factor (IGF), a leukemia inhibitory factor (LIF), Insulin, and Transferrin. According to some embodiments, the at least one growth factor is FGF2 or EGF2. According to some embodiments, the at least one growth factor is FGF2.

According to some embodiments, the concentration of the at least one growth factor in the first culture medium is from about 1 to about 100 ng/mL.

According to some embodiments, the first culture medium further comprises at least one Wnt inhibitor and/or activin A. According to some embodiments, the at least one Wnt inhibitor is selected from IWR1, XAV939, IWP-2, IWP-3, IWP-4, and iCRT3. According to some embodiments, the at least one Wnt inhibitor is IWR1. According to some embodiments, the concentration of the at least one Wnt inhibitor in the first culture medium is from about 1 μM to about 5 μM. According to some embodiments, the concentration of the activin A in the first culture medium is from about 10 ng/mL to about 50 ng/mL.

According to some embodiments, the culturing or the incubating at any of steps (a)-(c) further includes shaking of the cells.

According to some embodiments, the culturing or incubating at any of steps (a)-(c) is done in suspension.

According to some embodiments, the length of the first time period is from about 48 hours to about 96 hours.

According to some embodiments, the at least one BMP inhibitor is selected from LDN-193189, K02288, sclerostin, chordin, noggin, CTGF, follistatin, gremlin, inhibin, and BMP-3. According to some embodiments, the concentration of the BMP inhibitor in the second culture medium is from about 0.2 μM to about 1 μM.

According to some embodiments, the at least one Wnt activator is selected from Chir99021, Wnt3a, and Rspo3. According to some embodiments, the concentration of the Wnt activator in the second culture medium is from about 1 μM to about 15 μM.

According to some embodiments, the length of the second time period is from about 24 hours to about 96 hours.

According to some embodiments, step (b) further includes in intermediate step (b1), comprising replacing the second culture medium, after the cells have differentiated into posterior paraxial mesodermal organoids, with an intermediate culture medium of the same composition, or with an intermediate culture medium comprising a higher concentration of the at least one Wnt activator.

According to some embodiments, the concentration of the at least one Wnt activator in the third culture medium is from about 0.5 μM to about 10 μM.

According to some embodiments, the at least one hedgehog inhibitor is selected from GDC-0449, GANT58, and GANT-61. According to some embodiments, the concentration of the at least one hedgehog inhibitor in the third culture medium is from about 20 nM to about 500 nM.

According to some embodiments, the third culture medium further comprises one or more of Knock-out™ Serum Replacement (KSR), a growth factor, a BMP pathway activator, and an extracellular matrix (ECM) material. According to some embodiments, the concentration of the BMP pathway activator in the third culture medium is from about 10 ng/mL to about 100 ng/mL. According to some embodiments, the concentration of the KSR and/or the concentration of the ECM material in the third culture medium is about 1-20%.

According to some embodiments, the length of the third time period is from about 10 hours to about 48 hours.

According to some embodiments, the bPSCs express at least one of Oct4 and Sox2. According to some embodiments, the mesodermal organoids further express Sox2. According to some embodiments, the myogenic progenitor cells further express Pax7.

According to some embodiments, the bPSCs are cultured in step (a) at a density of about 5,000-100,000 cells/mL. According to some embodiments, each of the mesodermal organoids obtained in step (a) comprises, on average, about 50-300 bPSCs.

According to some embodiments, there is provide a method for producing bovine somite-like structures, the method comprising: (a) culturing bovine pluripotent stem cells (bPSCs) in a first culture medium comprising at least one growth factor for a first time period until aggregates form and the cells express Brachyury, thereby obtaining mesodermal organoids; and (b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and somite-like structures.

According to some embodiments, there is provided a method for generating myogenic progenitor cells from somite-like structures, the method comprising: (a) providing bovine somite-like structures in a culture medium; and (b) replacing the culture medium with a fresh culture medium comprising at least one Wnt activator and at least one hedgehog inhibitor and incubating until the cells express MyoD, thereby obtaining myogenic progenitor cells.

According to some embodiments, the somite-like structures are obtained by the methods of the invention.

According to some embodiments, there is provided an organoid comprising myogenic progenitor cells obtained by the methods of the invention.

According to some embodiments, there is provided a cultured meat product comprising the organoid of the invention.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1. Bovine mesodermal organoids formed at day 3. FIG. 1 presents an aggregate containing early mesodermal cells obtained from bovine ES cells (bESCs) following step (a) of the disclosed method. Shown are images of Brachyury-positive cells (BRA, red, AF2085-R&D Systems, top panel), Sox2 expressing cells (green, sc-365964, Santa Cruz, middle panel), and DAPI nuclear staining (blue, bottom panel). Scale bar=100 μm.

FIG. 2. Bovine posterior paraxial mesodermal organoids formed at day 5. FIG. 2 presents images of aggregates containing early paraxial mesodermal cells formed during step (b) of the disclosed method. Shown are DAPI nuclear staining (blue, top panel), Tbx6 expressing cells (an early paraxial mesoderm marker, green, ab38883, Abcam, middle panel), and of an overlap image (MERGED, bottom panel) of the two. Scale bar 100 uM. Aggregation method: shaking 24-well plate.

FIGS. 3A-3B. Bovine posterior paraxial mesodermal organoids formed at day 5. FIGS. 3A-B show aggregates containing early paraxial mesoderm and neural cells, obtained during step (b) of the disclosed method. (A) Images of bESC aggregates. (B) Images of bESC aggregates presenting a polarized pattern of Sox2 and Tbx6). Staining: Sox2 (pluripotent-neural, red, top left panel), Tbx6 (green, top right panel), DAPI (blue, bottom left panel), and an overlap image of the three (MERGED, bottom right panel). Scale bar 100 uM. Aggregation method: static 96-well U-bottom plate.

FIGS. 4A-4B. Bovine organoids at the anterior paraxial mesoderm stage. FIGS. 4A-B show aggregates containing matured paraxial mesoderm progenitors and somite-like structures, following step (b) of the disclosed method. (A) Images of organoids stained and fixed at day 5. No somite-like structures are observed. (B) Image of an example organoid stained and fixed at day 6. Somite-like structures are observed as closed-loop shapes of Pax3+ cells. Staining: Tbx6 (green, upper left panel), Pax3 (mature paraxial mesoderm, red, upper right panel, AB_528426, DSHB), DAPI (blue, middle panel), and of two overlap images: DAPI and Tbx6 (A) or DAPI, Pax3 and Tbx6 (B)(MERGED-1, lower left panel) and DAPI and Pax3 (MERGED-2, lower right panel). Scale bar 100 uM. Aggregation method: shaking 24-well plate.

FIG. 5A-5C The organoid differentiation process, days 3-6. (A) The differentiation protocol timeline during days 0-8 and the relevant factors at each step: days 0-3: FGF2, activin A, WNT inhibitor; days 3-5: BMP inhibitor and WNT activator; and days 5-8: WNT activator, BMP4, and hedgehog inhibitor. (B) Images of organoids at different days. Immunostaining (top to bottom): day 3: Oct4 (sc-8628, Santa Cruz, red), E-cadherin (24E10, Cell Signaling, blue), Sox2 (green, sc-365964, Santa Cruz), Brachyury (T, red, AF2085-R&D Systems), Tbx6 (green, ab38883, Abcam), and Sox17 (AF1924, R&D Systems, red); day 4: Tbx6, Pax3 (red, AB_528426, DSHB), Brachyury, and Sox2; day 5: Tbx6, Pax3, MyoD (red, PCRP-MYOD1-2A5-s, DSHB); day 6: Tbx6, Pax3, MyoD, and Pax7 (red, PAX7-s, DSHB). (C) Average percentage of positive cells within organoids (Y-axis) as a function of the differentiation time (days 3-8, X-axis), tested by multiple differentiation immunostainings (Sox2, Brachyury, Tbx6, Pax3, and MoyD), when cells are cultured with (IWR1+) or without (IWR1-) the addition of a Wnt inhibitor during days 0-3.

FIG. 6. The correlation between Pax3 expression and somite count. FIG. 6 presents the number of somites per organoid (Y-axis variable) Vs. the percentage of Pax3+ cells in organoids at Day 6 (X-axis variable), and the linear correlation between the two. Aggregation method: static U-bottom multi-well plates. Differentiation timeline days 0-3: including (IWR1+) and Activin A(+).

### DETAILED DESCRIPTION OF THE INVENTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

In the description and claims of the application, the words “contain”, “comprise”, “include” and “have”, and forms thereof, are not limited to members in a list with which the words may be associated.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

While cell-based meat science has evolved in the past few years, obtaining a large mass of edible bovine skeletal muscle tissue, is a major challenge. For this to be met, a scalable and efficient proliferation and muscle differentiation process is needed. While suspension culture methods offer the greatest promise for scaleup, they pose a hurdle toward muscle differentiation, due to the current need in physical anchoring points.

In brief, there are two main approaches for producing cultured meat: (1) starting from satellite cells—the relative benefit of cultured satellite cells is that their differentiation to muscle cells is comparatively fast and easy. However, as satellite cells are post mitotic (terminally differentiated) they do not proliferate and hence their use as the starting cells is not feasible for mass production; (2) starting from pluripotent cells (embryonic stem cells)—the benefit of using pluripotent cell is their outstanding long-term proliferative potential and their ability to be driven towards differentiate, upon being exposed to suitable conditions. However, the differentiation process of these cells to mature muscle cells takes very long time (over a month, usually, over 6 weeks).

A major challenge is the efficient growth of large mass of muscle cells, preferably within a tissue context, namely, cultured meat that resembles the contents of meat produce which is mainly composed of muscle tissue (about 90%) and further includes a combination of adipose (fat) cells, blood and connective tissue. The current solutions mostly rely on adult progenitor cells (e.g. satellite cells) as a starting point, and single cell type differentiation protocols, limiting both the proliferation capacity and the resemblance to full tissue structure. A scalable and efficient proliferation and muscle differentiation process is therefore required.

According to some embodiments, utilizing the protocols disclosed herein allows ultimately producing cultured meat that includes the various component of meat produce, primarily since the protocol produces bovine progenitor cells.

The term “protocol” as used herein is interchangeable with the term “method” or “process” in the context of a protocol for obtaining somite-like structures, myogenic progenitors, and 3D edible organoids.

Muscle differentiation protocols known to date are either based on 2D adherent cultures or are inefficient. Many protocols use serum and/or genetic modifications to activate certain key genes. Some use partially differentiated cells (such as satellite cells) as the starting cell type, which may place limits on the proliferation rate and capacity of these starting cells. The protocols disclosed herein combine signaling-only differentiation protocols, with suspended 3D culturing systems (starting from bovine embryonic stem cells (bESCs) resulting in an efficient muscle differentiation method which is scalable and free from serum and/or other animal-derived factors. Advantageously, the use of bovine pluripotent stem cells removes the need to repeatedly harvest the starting cell population from animals.

The protocols used for the preparation of the myogenic progenitor cells may be serum-free and/or genomic modification free, and may be based on one or more specific growing conditions, such as, mechanical-physiological conditions (including, for example, temperature, CO₂ concentration, cell density, agitation, etc.) and/or biochemical conditions (for example, media type, growth factors, inhibitors, activators, and the like).

The importance of using a serum-free medium is, inter alia, in prevention of infections, controlling the components of the medium, and preempting potential ethical issues.

According to some embodiments, provided herein is a novel organoid that recapitulates the developmental stages that cells undergo during embryonic development from pluripotent (ES) cells to skeletal muscle cells, in a 3D suspension organoid format. The organoid exhibits a spatial organization similar to the relevant structures in the developing embryo, and its growth protocol relies heavily on intra-organoid signaling. These properties make the protocol more amenable to adaptation to industrial scale.

The protocols provided in the present application are intended to be scalable and economical due to their suspension nature and use of intra-organoid signaling, while reducing the need for external factors in the culture medium. The protocols disclosed herein allow simulating the developmental stages and the spatial organization that cells undergo during embryonic development from bovine pluripotent cells to muscle progenitor cells, in a 3D suspension organoid format.

Advantageously, the simplified production process for bovine-cell derived cultivated meat products reduces not only the production cost but also the environmental impact. Further, in addition to its importance to cultured meat development, bovine organoids can also serve as a platform for drug/small compounds screenings for the animal health industry, thereby reducing animal experiments for these purposes, and significantly speeding up the development of medicine and food additives for the industrialized animal farming market.

In view of the foregoing, according to some embodiments, there is provided a scalable, minimally directed method for growing bovine muscle organoids starting from bovine ESCs, in an economically viable process. First, bESCs as source cells allow higher amount and faster population doublings (compared to adult progenitor cells, such as satellite cells) at the proliferation stage. bESC and/or bovine induced pluripotent stem cells (biPSC), together termed herein “bovine pluripotent stem cells (bPSCs)” lines may be used as source cells for the organoid of the invention. The bESCs and/or biPSCs cells are adapted to suspension growth, as aggregates. Second, such a process allows to unify the proliferation and differentiation stages into a single bioreactor format. Finally, the in-organoid spontaneous differentiation process leads to a mixture of cell types, thereby alleviating the need for separate differentiation processes while reducing external differentiation factors usage.

The terms “aggregates”, “3D organoids” and “organoids” as used herein, are interchangeable.

Accordingly, the bovine organoid system can provide a scalable, economical approach for generating bovine muscle progenitor cells from bESCs. The established protocols may thereafter be readily adopted for the production of cultivated meat in scaled-up bioreactors. Further, the system can significantly improve scalability and reproducibility of muscle tissue production, reduce costs of production, making mass scale commercialization more feasible and faster.

According to some embodiments, there is provided herein a developmental-path based serum-free, genomic modifications free, protocol for the generation, in culture, of bovine somites or myogenic progenitor cells. The protocol may further be expanded to generate muscle cells and muscle tissue. The protocol includes inducing muscle differentiation within 3D suspended bovine cell cultures, starting from bovine embryonic stem cells. Further provided are cultured bovine myogenic progenitor cells, and optionally bovine somite-like structures which include muscle cells.

The protocols disclosed herein which can be used for the preparation of bovine meat-like products in culture, combine self-organization properties of embryonic 3D developmental system at the micro scale, with biotechnological tools, which render them highly suitable for mass production of bovine meat products and for muscle tissue maturation within organoid cultures. The protocol cures the large gap between existing established bovine muscle differentiation protocols and the scaleup and safety requirements for food production.

Surprisingly, the protocols enable the production of bovine myogenic progenitor cells, which are enclosed structures that give rise to vertebrae and skeletal muscle in the embryo. The in-vitro production of bovine myogenic progenitor cells has major benefits. First, production of myogenic progenitor cells indicates that the protocol (method/process) well mimics/simulates the developmental stages that cells undergo during embryonic development from pluripotent (ES) bovine cells to fully differentiated bovine skeletal muscle cells, in a 3D suspension organoid format. As a result, the cultured tissue produced herein Are expected to include all the feature of ‘real’ bovine meat, namely, it is primarily made of bovine muscle tissue but may also include other related tissues, such as, adipose cell, connective tissue, and blood, contributing to the structure and taste of meat produced from livestock. Second, the presence of myogenic progenitor cells in the production process results in intra-organoid signaling, which promotes the required differentiation independently of external supplements, and hence only little amounts of growth factors are used for supplementing the culture medium. Accordingly, the protocol disclosed herein is cost-effective, since it does not require large quantities of costly growth factors (that typically create the major expenditure in the production of cultured tissues).

According to some embodiments, the cell type composition in the organoid may be characterized at different steps of the organoid differentiation process (protocol).

According to some embodiments, efficient aggregation protocols for bESC/biPSCs, allowing controlled initial aggregate size and preserving the differentiation potential are provided. In further embodiments, there is provided an optimized protocol for generating bESC-derived mesodermal organoids, with a polarized pattern. Characterization of organoid cell composition and spatial structure may be performed by suitable methods, such as, for example, by immunostaining, FISH and qPCR. In additional embodiments, there are provided methods leading to the bovine myogenic progenitor cells stage starting from bovine cell lines. In some embodiments, further provided are high-resolution characterizations of the cell types within the bovine organoid, their proportions, their spatial organization as well as quantification of somite number distribution within organoids.

According to some embodiments, the methods presented herein initiate with bovine stem cells and enable differentiation in suspension (e.g. in large scale bioreactors or even in conventional steerers), advantageously until obtaining bovine myogenic progenitor cells.

According to some embodiments, the protocols are fast, cost-effective, reproducible, and suitable for large scale bioreactors, while allowing the production of cultured meat that includes the feature of livestock meat.

According to some embodiments, there is provided a process for the generation of 3D organoids from bovine ESC (bESC). To this aim, growth and aggregation conditions for bESC lines are used, while maintaining their differentiation potential and adaptation to suspension. In some embodiments, optimized conditions for directing the bESC-derived aggregates toward mesodermal lineages, are used. According to further embodiments, the self-organization properties of the 3D developmental system and the high proliferation capacity of bESCs are used to enable mass production of bovine muscle precursor organoids, which can be then combined and matured through integration with a scaffolding system for the formation of cultivated meat products.

The disclosed protocol is composed of a series of steps, where each step attempts to efficiently mimic/simulate in-vivo signaling environment sensed by the myogenic lineage at the relevant developmental stage, e.g. mesoderm, paraxial mesodermal, myogenic progenitor cells and mature muscle cells. The first step of the protocol includes aggregating bovine ES cells under a first culture medium, following the differentiation of the aggregate in suspension, where the first culture medium is modified between some consecutive steps.

According to some embodiments, there is provided a method for generating bovine myogenic progenitor cells, the method comprising:
(a) culturing bovine pluripotent stem cells (bPSCs) in a first culture medium comprising at least one growth factor for a first time period until aggregates form and the cells express Brachyury, thereby obtaining mesodermal organoids;
(b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and optionally somite-like structures;
(c) replacing the second culture medium with a third culture medium comprising at least one Wnt activator and at least one hedgehog inhibitor and incubating for a third time period until the cells express MyoD, thereby obtaining myogenic progenitor cells.

In some embodiments, the bPSCs are derived from a bovine selected from cattle, bison, buffalo, or antelope. In some embodiments, the bPSCs are derived from bovine cattle such as a cow or a bull. In some embodiments, the bPSCs are derived from a cow.

The bPSCs may be any suitable bovine pluripotent stem cells. In some embodiments, the bPSCs are selected from bovine embryonic stem cells (bESCs) and bovine induced pluripotent stem cells (biPSCs). In some embodiments, the bPSCs are bESCs. In some embodiments, the bPSCs are biPSCs.

Pluripotent stem cells can be defined by pluripotency characteristics including, but not limited to: (i) pluripotent stem cell morphology; (ii) the potential for unlimited self-renewal; (iii) expression of pluripotent stem cell markers including, but not limited to: OCT4, SOX2, NANOG; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The terms “pluripotent stem cell” and “embryonic stem cell” as used herein are interchangeable. In some embodiments, the bPSCs used herein are free of genetic engineering.

The term “culturing” as used herein refers to culturing, propagation, or growth in a 2D culture or a 3D suspension under conditions that are suitable for proliferation. When 3D growth is indicated implicitly or explicitly (e.g. when referring to “suspension”), then the growth is in a 3D suspension. Cell proliferation generally increases the size of the aggregates forming larger aggregates, which can be routinely mechanically or enzymatically dissociated into smaller aggregates to maintain cell proliferation within the culture and increase numbers of cells.

According to some embodiments, at least some of the steps may be carried out on culture plates (e.g. Petri dishes and/or multi-well plates).

The bPSCs may be seeded for culturing at any appropriate density. In some embodiments, the cells are cultured at a density of about 5,000 to about 100,000 cell/mL, about 10,000 to about 100,000 cell/mL, about 20,000 to about 100,000 cell/mL, about 50,000 to about 100,000 cell/mL, about 10,000 to about 70,000 cell/mL, about 20,000 to about 70,000 cell/mL, about 50,000 to about 70,000 cell/mL, about 10,000 to about 50,000 cell/mL, about 20,000 to about 50,000 cell/mL, about 40,000 to about 60,000 cells/mL, about 50,000 to about 55,000 cells/mL, or about 50,000 cell/mL. Each possibility is a different embodiment. In some embodiments, the cells are cultured at a density of about 50,000 cell/mL.

According to some embodiments, the culture media, including the first, second, third, and/or fourth culture media may include any culture medium suitable for use with bovine pluripotent stem cells. According to some embodiments, any of the culture media used herein, such as the first, second, third, and/or fourth culture media may include N2B27 culture medium, NFBR culture medium (Soto et al., Scientifc Reports (2021) 11:11045), neural differentiation medium (NDIFF®), mTeSR™1 Pluripotent Stem Cell (PSC) Maintenance medium, or similar. In some embodiments, the culture medium is replaced between the steps with the same culture medium or with a different culture medium, according to the respective step. In some embodiments, each of the culture media includes a different reagent/plurality of reagents, according to the step of the protocol. In some embodiments, at any step, instead of replacing a certain culture medium with the next culture medium, only the desired ingredients are added, and any undesired ingredients are removed.

In some embodiments, any of the culture media are serum-free. In some embodiments, all of the culture media are serum-free.

According to some embodiments, any of the culture media used herein, such as the first, second, third, and/or fourth culture media, may be supplemented with Knock-out™ Serum Replacement (KSR). The KSR helps the cells thrive in the serum-free medium.

In some embodiments, the concentration of the KSR in any of the first, second, third, or fourth culture medium is from about 1 to about 10%, about 3 to about 10%, about 5 to about 10%, about 1 to about 7%, about 3 to about 7%, about 5 to about 7%, about 1 to about 5%, about 2 to about 5%, about 3 to about 5%, or about 5%. Each possibility is a separate embodiment. In some embodiments, the concentration of the KSR in the respective culture medium is about 5%.

In some embodiments, the at least one growth factor is selected from a fibroblast growth factor (FGF), an epidermal growth factor (EGF), a hepatocyte growth factor (HGF), an insulin-like growth factor (IGF), a leukemia inhibitory factor (LIF), Insulin, and Transferrin. In some embodiments, the at least one growth factor is FGF2 or EGF2. In some embodiments, the at least one growth factor is FGF2.

In some embodiments, the concentration of the at least one growth factor in the first culture medium is about 1 to about 200 ng/mL, about 5 to about 100 ng/mL, about 10 to about 100 ng/mL, about 10 to about 50 ng/mL, about 20 to about 50 ng/mL, about 20 ng/mL to about 30 ng/mL, or about 20 ng/mL. Each possibility is a separate embodiment. In some embodiments, the concentration of the at least one growth factor in the first culture medium is about 20 ng/mL.

According to some embodiments, at any of steps (a)-(c) the cells are grown, cultured, or incubated in suspension. In other words, the cells may be suspended in any of the culture media, i.e. the first, second, third, or fourth culture media. In some embodiments, the cells are grown in suspension throughout the whole method, until becoming myogenic progenitors, or until becoming myocytes. In some embodiments, the cells are initially grown in a 2D culture such as on feeder cells, and are detached and become suspended in the respective culture medium during the method, for example, during step (a), during step (b), or during step (c).

According to some embodiments, any of steps (a)-(c) further comprise shaking, or agitating, the cells. According to some embodiments, any of steps (a)-(d) further comprise shaking the cells. According to some embodiments, step (a) further comprises shaking the cells. According to some embodiments, step (b) further comprises shaking the cells. According to some embodiments, step (c) further comprises shaking the cells. According to some embodiments, step (d) further comprises shaking the cells. According to some embodiments, shaking of the cells starts at step (a) and continues until myogenic progenitor cells, or until myocytes, are obtained. According to some embodiments, shaking of the cells starts at step (b) and continues until myogenic progenitor cells, or until myocytes, are obtained. According to some embodiments, shaking of the cells starts at any of the steps in the method, and continues until myogenic progenitor cells, or until myocytes are obtained. In some embodiments, shaking is conducted in step (a) to enhance forming aggregates.

In some embodiments, the shaking is conducted in an orbital shaker. In some embodiments, the shaking speed depends on the density of the cells and the circumference of the plates, or the wells in which the cells are seeded or cultured. For example, for aggregating cells seeded at a density of about 50,000 cells/mL in 24-well plates, the shaking speed is about 150 rpm.

Accordingly, in some embodiments, the shaking speed is about 50 to about 500 rpm, about 100 to about 400 rpm, about 100 to about 300 rpm, about 100 to about 200 rpm, or about 150 rpm. Each possibility is a separate embodiment. In some embodiments, the shaking speed is about 150 rpm.

In some embodiments, aggregates are formed without shaking. In some embodiments, the method is conducted without shaking. For example, in some embodiments, cells are seeded as single droplets in a low-adherence U-bottom plate at a concentration of about 10,000-50,000 or about 12,500-25,000 cells/mL.

According to some embodiments, each of the mesodermal organoids, or aggregates, obtained in step (a) comprises, on average, about 10 to about 1000, about 20 to about 1000, about 50 to about 1000, about 10 to about 500, about 20 to about 500, about 50 to about 500, about 10 to about 300, about 20 to about 300, or about 50 to about 300 bPSCs. Each possibility is a separate embodiment. According to some embodiments, each of the mesodermal organoids, or aggregates, obtained in step (a) comprise, on average, about 50-300 bPSCs.

According to some embodiments, the aggregates are essentially spheric. In some embodiments, a plurality of spheric aggregates is obtained.

According to some embodiments, the plurality of spheric aggregates obtained at the end of step (a) include at least 50 spheric aggregates, at least 100 spheric aggregates, at least 150 spheric aggregates, at least 200 spheric aggregates, at least 250 spheric aggregates, at least 300 spheric aggregates, or about 300 spheric aggregates. Each possibility represents a separate embodiment.

It is noted that cells cultured within aggregates in maintenance culture generally maintain markers of pluripotency, as can be determined, in part, by assessing pluripotency characteristics of the cells, as explained above, with reference to bPSCs. The pluripotent stem cell aggregates were expected to require further differentiation cues to induce differentiation.

Therefore, it was a very surprising finding that the suspended organoids differentiated, in step (a), into a mesodermal fate, without the aid of additional differentiation factors.

It is noted that pluripotent stem cell morphology has classical morphological features of an embryonic stem cell, and normal embryonic stem cell morphology may be characterized by being round (spheric) and small in shape, with a high nucleus-to-cytoplasm ratio, the notable presence of nucleoli, and typical inter-cell spacing. In contrast, aggregates comprising mesodermal may have ovoid-like morphology.

Furthermore, it has been surprisingly shown, that the first step of the protocol, which includes aggregating bESCs which further differentiate into mesodermal organoids, can be performed in the presence of a Wnt inhibitor, which is normally required in order to maintain pluripotency.

Accordingly, in some embodiments, the first culture medium further comprises at least one Wnt inhibitor, at least one ROCK inhibitor, and/or activin A.

According to some embodiments, the at least one Wnt inhibitor is selected from IWR1, XAV939, IWP-2, IWP-3, IWP-4, and iCRT3. According to some embodiments, the at least one Wnt inhibitor is IWR1.

According to some embodiments, the concentration of the Wnt inhibitor in the first culture medium is about 0.7 to about 10 μM, about 0.7 to about 7 μM, about 1 to about 7 μM, about 1 to about 5 μM, about 1 to about 3 μM, about 2 to about 3 μM, or about 2.5 μM. Each possibility represents a separate embodiment. According to some embodiments, the concentration of the Wnt inhibitor in the first culture medium is about 2.5 μM.

In some embodiments, the concentration of activin A in the first culture medium is about 10 to about 100 ng/mL, about 10 to about 50 ng/mL, about 20 to about 50 ng/mL, about 10 to about 30 ng/mL, about 20 to about 30 ng/mL, or about 20 ng/mL. Each possibility represents a separate embodiment. In some embodiments, the concentration of Activin A in the first culture medium is about 20 ng/mL.

In some embodiments, the at least one ROCK inhibitor is selected from Y-27632 (Dihydrochloride), WAY-624704, Thiazovivin, GSK429286A, or any other suitable ROCK inhibitor, and is added at a concentration of about 0.5-200 μM, 1-100 μM, 5-50 μM, or 10-20 μM.

According to the present invention, the cell differentiation state at different stages of the process is characterized using landmark gene markers, such as Brachyury (T, or TBXT) (for mesodermal organoids), TBX6 and/or MSGN1 (for posterior paraxial mesoderm), PAX3 (for anterior paraxial mesoderm), optionally UNCX, TBX18 and/or TCF15 (for somite-like structures), and MyoD and/or Pax7 for myogenic progenitor cells. Such characterization may be carried out by any suitable method. In some embodiments, mRNA profiling using FISH, and antibody staining can be utilized for such characterization. Such identified markers may be imaged in 3D using suitable imaging systems (such as, a two-photon microscopy system). Additionally, qPCR may be performed on pooled organoids, on a panel of genes representing such structures.

The mesodermal organoids obtained in the end of step (a) are defined by the expression of Brachyuri. In some embodiments, the mesodermal organoids further express Sox2.

According to some embodiments, the length of the first time period is about 10 to about 96 hours, about 24 to about 96 hours, about 48 to about 96 hours, about 24 to about 84 hours, about 48 to about 84 hours, about 24 to about 72 hours, about 48 to about 72 hours, at least two days, at least 48 hours, at least 72 hours, about 1 day, about 2 days, about 3 days, about 48 hours, or about 72 hours. Each possibility represents a separate embodiment.

In some embodiments, the method further includes an additional step, preceding step (a), which includes culturing the bPSCs in the first medium in a 2D culture with or without feeder cells. If bPSCs are cultured on a feeder layer, then at the next step they are detached from the feeder cells. The feeder cells may be any suitable feeder cells, for example, MEF feeder cells. The detaching from the feeder cells may be conducted by any suitable method, including but not limited to digestion by trypsin (e.g. TrypLE™). The free bPSCs are used in step (a) of the method, disclosed herein.

In some embodiments, the at least one BMP inhibitor is selected from LDN-193189, K02288, sclerostin, chordin, noggin, CTGF, follistatin, gremlin, inhibin, and BMP-3. In some embodiments, the at least one BMP inhibitor is LDN-193189.

In some embodiments, the concentration of the BMP inhibitor in the second culture medium is from about 0.1 μM to about 2 μM, from about 0.1 μM to about 1 μM, from about 0.1 μM to about 0.7 μM, from about 0.1 μM to about 0.5 μM, from about 0.2 μM to about 2 μM, from about 0.2 μM to about 1 μM, from about 0.2 μM to about 0.7 μM, from about 0.2 μM to about 0.5 μM, from about 0.3 μM to about 1 μM, from about 0.3 μM to about 0.7 μM, from about 0.3 μM to about 0.5 μM, from about 0.4 μM to about 0.6 μM, or about 0.5 μM. Each possibility is a separate embodiment. In some embodiments, the concentration of the BMP inhibitor in the second culture medium is about 0.5 μM.

In some embodiments, the at least one Wnt activator is selected from Chir99021, Wnt3a, and Rspo3. In some embodiments, the at least one Wnt activator is Chir99021. In some embodiments, the at least one Wnt activator is Rspo3. In some embodiments, the at least one Wnt activator is Wnt3a.

In some embodiments, the concentration of the Wnt activator in the second culture medium is from about 1 to about 20 μM, about 1 to about 15 μM, about 1 to about 10 μM, about 2 to about 20 μM, about 2 to about 15 μM, about 2 to about 10 μM, about 5 to about 20 μM, about 5 to about 15 μM, about 5 to about 10 μM, about 7 to about 20 μM, about 7 to about 15 μM, about 7 to about 10 μM, or about 10 μM. Each possibility represents a separate embodiment. In some embodiments, the concentration of the Wnt activator in the second culture medium is about 10 μM.

The term “essentially devoid of”, as used herein with reference to ingredients excluded from a culture medium, means either that the respective culture medium does not contain any of the excluded ingredients (e.g., the second culture medium does not contain any Wnt inhibitor and any activin A), or that any of the excluded ingredients are present at a level low enough not to cause any of the effects expected from the excluded ingredients. Accordingly, a medium devoid of Wnt inhibitor may include up to 0.5 μM of Wnt inhibitor, and/or a medium devoid of activin A may include up to 5 ng/ml activin A.

In some embodiments, instead of replacing the first culture medium with the second culture medium, the added ingredients of the second culture medium are added (e.g. BMP inhibitor and Wnt activator), and any Wnt inhibitor and/or activin A present in the first culture medium are removed.

According to some embodiments, the addition of at least one Wnt activator and at least one BMP inhibitor to the second culture medium at step (b) reduce the overall viscosity of the culture medium thereby facilitating growth in a mixed suspension condition, which is especially suitable for industrial scale bioreactors.

In some embodiments, the growth factor used in the second culture medium is the same growth factor used in the first culture medium. In some embodiments, the growth factor used in the second culture medium is not the same as the growth factor used in the first culture medium.

In some embodiments, the concentration of the growth factor used in the second culture medium is the same as that used in the first culture medium. In some embodiments, the concentration of the growth factor used in the second culture medium is not the same as that used in the first culture medium.

According to some embodiments, the length of the second time period is about 24 to about 96 hours, about 36 to about 96 hours, about 48 to about 96 hours, about 72 to about 96 hours, about 24 to about 84 hours, about 36 to about 84 hours, about 48 to about 84 hours, about 72 to about 84 hours, about 24 to about 72 hours, about 36 to about 72 hours, about 48 to about 72 hours, about 48 to about 84 hours, or about 72 hours. Each possibility is a separate embodiment. According to some embodiments, the length of the second time period is about 72 hours.

The anterior paraxial mesodermal organoids obtained in the end of step (b) are defined by the expression of Tbx6 and Pax3.

The term “somite-like structures”, as used herein, is used interchangeably with “somites”. Somite-like structures may be identified by detecting expression of UNCX, TBX18 and/or TCF15, and/or by their epithelium-surrounded lumen shapes. In some embodiments, somite-like structures are identified as closed loops of Pax3-positive cells.

In some embodiments, step (b) further includes an intermediate step (b1), comprising replacing the second culture medium, after the cells have differentiated into posterior paraxial mesodermal organoids but not yet into anterior paraxial mesodermal organoids, with an intermediate culture medium.

In some embodiments, the posterior paraxial mesodermal organoids are identified by expression of Tbx6, but no (or low) expression of Pax3.

In some embodiments, step (b1) is conducted after about 10 hours to about 72 hours, about 10 hours to about 48 hours, about 10 hours to about 36 hours, about 10 hours to about 24 hours, about 24 hours to about 72 hours, about 24 hours to about 48 hours, about 24 hours to about 36 hours, or about 48 hours, after the addition of the second culture medium. Each possibility is a separate embodiment.

In some embodiments, the intermediate culture medium comprises a higher concentration of Wnt activator than the concentration of the Wnt activator in the second culture medium. Alternatively, instead of replacing the medium in step (b1), a Wnt activator is added to the culture.

According to some embodiments, the at least one Wnt activator used in the third culture medium is the same as the at least one Wnt activator used in the second culture medium. According to some embodiments, the at least one Wnt activator used in the third culture medium is not the same as the at least one Wnt activator used in the second culture medium.

According to some embodiments, the concentration of the at least one Wnt activator in the third culture medium is lower than the concentration of the at least one Wnt activator in the second culture medium.

According to some embodiments, the concentration of the at least one Wnt activator in the third culture medium is from about 0.5 μM to about 20 μM, about 1 μM to about 20 μM, about 2 μM to about 20 μM, about 3 μM to about 20 μM, about 0.5 μM to about 10 μM, about 1 μM to about 10 μM, about 2 μM to about 10 μM, about 3 μM to about 10 μM, about 0.5 μM to about 7 μM, about 1 μM to about 7 μM, about 2 μM to about 7 μM, about 3 μM to about 7 μM, about 0.5 μM to about 5 μM, about 1 μM to about 5 μM, about 2 μM to about 5 μM, about 3 μM to about 5 μM, about 0.5 μM to about 3 μM, about 1 μM to about 3 μM, about 2 μM to about 3 μM, about 2 μM to about 4 μM, or about 3 μM. Each is a separate embodiment. According to some embodiments, the concentration of the at least one Wnt activator in the third culture medium is about 3 μM.

According to some embodiments, the at least one hedgehog inhibitor is selected from GDC-0449, GANT58, and GANT-61. According to some embodiments, the at least one hedgehog inhibitor is GDC-0449.

According to some embodiments, the concentration of the at least one hedgehog inhibitor in the third culture medium is from about 20 nM to about 500 nM, 50 nM to about 500 nM, 100 nM to about 500 nM, 150 nM to about 500 nM, 20 nM to about 300 nM, 50 nM to about 300 nM, 100 nM to about 300 nM, 150 nM to about 300 nM, 20 nM to about 200 nM, 50 nM to about 200 nM, 100 nM to about 200 nM, 150 nM to about 200 nM, or about 150 nM. Each is a separate embodiment. According to some embodiments, the concentration of the at least one hedgehog inhibitor in the third culture medium is about 150 nM.

According to some embodiments, the third culture medium further comprises one or more of KSR, a growth factor, a BMP pathway activator, an extracellular matrix (ECM) material, a micro-scaffolds, or protein fibers/filaments. According to some embodiments, the third culture medium further comprises a BMP pathway activator.

In some embodiments, the concentration of the KSR in the third culture medium is from about 1 to about 20%, about 3 to about 20%, about 5 to about 20%, about 10 to about 20%, about 1 to about 10%, about 3 to about 10%, about 5 to about 10%, about 1 to about 7%, about 3 to about 7%, about 5 to about 7%, about 1 to about 5%, about 2 to about 5%, about 3 to about 5%, or about 5%. Each possibility is a separate embodiment. In some embodiments, the concentration of the KSR in the third culture medium is about 5%.

In some embodiments, the growth factor used in the third culture medium is the same growth factor used in the first culture medium. In some embodiments, the growth factor used in the third culture medium is not the same as the growth factor used in the first culture medium.

In some embodiments, the concentration of the growth factor used in the third culture medium is the same as that used in the first culture medium. In some embodiments, the concentration of the growth factor used in the third culture medium is not the same as that used in the first culture medium.

In some embodiments, the growth factor is selected from HGF, IGF, FGF2, and insulin. In some embodiments, the HGF concentration in the third medium is about 1-75 ng/mL. In some embodiments the IGF concentration in the third medium is about 0.1-20 ng/mL. In some embodiments the FGF2 concentration in the third medium is about 1-200 ng/mL. In some embodiments the insulin concentration in the third medium is about 0.1-15%. In some embodiments, the concentration of the growth factor in the third medium is less than about 30 ng. In some embodiments, the concentration on the growth factor in the third medium is within the range of about 1 to 25 ng/mL.

In some embodiments, the BMP pathway activator is BMP4.

In some embodiments, the concentration of BMP pathway activator in the third culture medium is from about 10 ng/mL to about 100 ng/mL, about 10 ng/mL to about 80 ng/mL, about 10 ng/mL to about 50 ng/mL, about 30 ng/mL to about 100 ng/mL, about 30 ng/mL to about 80 ng/mL, about 30 ng/mL to about 50 ng/mL, about 50 ng/mL to about 100 ng/mL, about 50 ng/mL to about 80 ng/mL, about 40 ng/mL to about 60 ng/mL, or about 50 ng/mL. Each possibility is a separate embodiment. In some embodiments, the concentration of BMP pathway activator in the third culture medium is about 50 ng/mL.

The term “ECM material” relates to a natural component of the extracellular matrix, or a synthetic material intended to function as ECM. In some embodiments, the extra cellular matrix (ECM) material is Matrigel or an ECM component.

In some embodiments, the ECM component is selected from fibronectin, collagen, and laminin.

In some embodiments, the Matrigel concentration in the third culture medium is about 1-20%.

In some embodiments, the micro-scaffolds or protein fibers/filaments are SpheroSeev or mycelium.

According to some embodiments, the concentration of the growth factors in the third medium is about 0.5-100 ng/mL. According to some embodiments, the concentration of the Wnt activator in the third medium is about 0.5-20 μM. According to some embodiments, the concentration of the Hedgehog inhibitor in the third medium is in the range of about 25-500 nM.

According to some embodiments, the concentration of the KSR in the third medium is about 1-20%. According to some embodiments, the concentration of the ECM in the third medium is about 1-20%.

According to some embodiments, the myogenic progenitor cells further express Pax7.

According to some embodiments, the obtained myogenic progenitor cells further include myoblasts, myocytes and satellite cells.

According to some embodiments, the length of the third time period is about 10 to about 72 hours, about 10 to about 48 hours, about 10 to about 36 hours, about 10 to about 24 hours, about 20 to about 72 hours, about 20 to about 48 hours, about 20 to about 36 hours, about 20 to about 24 hours, about 24 to about 72 hours, about 24 to about 48 hours, about 24 to about 36 hours, about 20 to about 30 hours, at least one day, at least two days, or about 24 hours. Each possibility is a separate embodiment. According to some embodiments, the length of the third time period is about 24 hours.

In some embodiments, the method includes a further step, which comprises differentiation of the myogenic progenitor cells obtained in step (c) to myocytes and muscle fibers.

In some embodiments, step (d) includes replacing the third culture medium with a fourth culture medium, and incubating for about 10-72 hours, until myocytes are identified.

According to some embodiments, the fourth culture medium comprises one or more of: a Wnt activator, KSR, a growth factor, a BMP pathway activator, an extra cellular matrix (ECM) material, or micro-scaffolds or protein fibers/filaments, and is essentially devoid of a hedgehog inhibitor. Each possibility is a separate embodiment.

In some embodiments, the Wnt activator is at a concentration of about 0.5-10 μM in the fourth medium. In some embodiments, the KSR is at a concentration of about 0.5-20% in the fourth medium. In some embodiments, the growth factor is selected from HGF, IGF, FGF2, and insulin. In some embodiments, the HGF is at a concentration of about 2-50 ng/mL in the fourth medium. In some embodiments the IGF is at a concentration of about 0.5-10 ng/mL in the fourth medium. In some embodiments the FGF2 is at a concentration of about 2-100 ng/mL in the fourth medium. In some embodiments the insulin is at a concentration of about 0.5-5% in the fourth medium. In some embodiments, the concentration of the one or more growth factor in the fourth medium is less than about 30 ng. In some embodiments, the concentration on the one or more growth factor in the fourth medium is within the range of about 1 to 25 ng/mL.

In some embodiments, the extra cellular matrix (ECM) material is Matrigel or an ECM component. In some embodiments, the ECM component is selected from fibronectin, collagen, and laminin. In some embodiments, the Matrigel concentration is about 1-20%. In some embodiments, the micro-scaffolds or protein fibers/filaments are SpheroSeev or mycelium.

According to some embodiments, the concentration of the growth factors in the fourth medium is in the range of about 0.5-100 ng/mL. According to some embodiments, the concentration of the Wnt activator in the fourth medium is in the range of about 0.5-20 μM. According to some embodiments, the concentration of the KSR in the fourth medium is about 1-20%. According to some embodiments, the concentration of the ECM material in the fourth medium is about 1-20%.

According to some embodiments, the length of the fourth time period is about 10-72 hours. For example, 24-48 hours, at least one day, about 1-2 days, or at least two days. Each possibility is a separate embodiment.

It is noted that any protein used in any of the first, second, third, or fourth culture media, may be produced by any suitable method, including by recombinant technology. Accordingly, any of the protein factors used in the invention may be a recombinant protein.

In some embodiments, the aforesaid steps (a)-(b) are used to produced somites, or somite-like structures. These steps may be followed immediately or at a different time by the aforesaid step (c) to give rise to myogenic progenitor cells. Step (c) may then be followed by the aforesaid step (d), to give rise to myocytes.

Therefore, according to some embodiments, there is provided a method for producing somite-like structures, comprising:
(a) culturing bovine pluripotent stem cells (bPSCs) in a first culture medium comprising at least one growth factor for a first time period until aggregates form and the cells express Brachyury, thereby obtaining mesodermal organoids; and
(b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and somite-like structures.

According to some embodiments, any of steps (a) and (b) of the above method for producing somite-like structures are done in the same way as any of the embodiments of the respective steps (a) and (b) of the method for generating bovine myogenic progenitor cells disclosed hereinabove.

According to some embodiments, there is provided a method for generating myogenic progenitor cells from somite-like structures, the method comprising:
(a) providing bovine somite-like structures in a culture medium; and
(b) replacing the culture medium with a fresh culture medium comprising at least one Wnt activator and at least one hedgehog inhibitor and incubating until the cells express MyoD, thereby obtaining myogenic progenitor cells.

In some embodiments, the bovine somite-like structures are obtained by the methods of the invention.

According to some embodiments, step (b) of the above method for generating myogenic progenitor cells from somite-like structures is done in the same way as any of the embodiments of step (c) of the method for generating bovine myogenic progenitor cells disclosed hereinabove.

In some embodiments, the culture medium is the second culture medium of the invention, and the fresh culture medium is the third culture medium of the invention.

In some embodiments, the incubating is for the third time period disclosed in the invention.

In some embodiments, there is provided an organoid comprising myogenic progenitor cells obtained by the methods described herein.

In some embodiments, there is provided a cultured meat product comprising the organoid disclosed herein.

According to some embodiments, a scaffolding (extracellular) matrix, or components thereof, may be incorporated into the aggregates in the suspension. According to some embodiments, the culture medium comprises low concentrations of extracellular matrix (ECM) or components thereof, specifically, less than about 20% vol/vol, less than 15% vol/vol, less than 10% vol/vol, less than 8% vol/vol, less than 5% vol/vol. According to some embodiments, the ECM comprise Matrigel™. According to some embodiments, the culture medium comprises components of the ECM. According to some embodiments, the components of the ECM include, but are not limited to, fibronectin, collagen and laminin.

According to some embodiments, the addition of ECM or components thereof is performed after observing aggregates having ovoid-like morphology. Thus, the protocol is not based on random/constant timing for adding the ECM or its components, since it has been found that the right timing changes from batch to batch, and surprisingly, it is best to add ECM or its components based on observation of the aggregates, wherein the ideal timing is when the aggregates present an ovoid-like morphology.

Following the addition of ECM or components thereof, paraxial mesodermal cells are formed. According to some embodiments, the ECM or components thereof are removed from the culture medium following the formation of paraxial mesodermal cells within the aggregates. Removal of ECM or its components from the culture medium confers an additional advantage to the disclosed protocol: it reduces the overall cost of the entire protocol while not compromising on the desired outcome.

According to some embodiments, the ECM or components thereof are maintained in the culture medium.

Maturation of muscle cells has been demonstrated to depend on anchoring of the cells on (semi-)rigid surfaces, likely mimicking anchoring to bone structures. Accordingly, in some embodiments, the method disclosed herein may further includes the step of embedding the late-stage organoids in an edible matrix, in an attempt to generate anchor points of the maturation of myocytes. Optional matrices include, but are not limited to, mycelium, alginate and cellulose (e.g. from decellularized apples).

According to some embodiments, the organoids are embedded at the end of the differentiation process, into a scaffolding matrix.

According to some embodiments, the method further comprises monitoring differentiation in the suspended 3D-organoids.

According to some embodiments, the monitoring is carried out using at least one technique selected from: high throughput two-photon 3D imaging, live imaging, immunostaining against marker proteins, RT-PCR and FISH.

Imaging is also used to monitor the cell composition at the different phases of the process for the purpose of identifying efficiency bottlenecks in terms of the fraction of muscle lineage cells in the organoid.

According to some embodiments, the method further includes characterizing the cell populations in the 3D-organoids at different time points.

According to some embodiments, the method further includes characterizing the spatial organization of the cell populations in the 3D-organoids at different time points.

According to some embodiments, characterizing the cell populations in the 3-D organoids is carried out by various methods, including, but not limited to, RT-PCR and FISH.

According to some embodiments, the method may further include nutritional profiling the 3-D organoids. For nutritional profiling various approaches can be applied, such as, GC, HPLC, and GC-MS with cold EI.

According to some embodiments, the 3D organoids may be in a format such as hanging drops, rotating suspension, free suspension, patterned microwells and high-throughput 96-wells.

According to some embodiments, the method includes adding in one or more of the steps one or more of: A23187, calcium, external protein fibers (such as SpheroSeev and mycelium) in various densities between the organoids. Such agents may aid self-assembly and muscle fiber orientation of the whole tissue, increase muscle fibers yield (by serving as anchor surfaces), and reduce necrosis (by increasing the porosity of the tissue, enabling better access to oxygen and nutrients).

According to some embodiments, there is provided a scalable, end-to-end production process for the generation of whole-cut skeletal muscle tissue, using bESC-based muscle organoids as building blocks. These organoids are generated in suspension through a developmentally inspired protocol and are differentiated from bESCs to muscle progenitor organoids. These provide a scalable and economical approach to generate myogenic progenitor cells in suspension and reducing the amounts of externally added growth factors. Culture conditions are utilized to allow intra-organoid muscle maturation and then inter-organoid muscle fiber fusion and self-assembly, mimicking myogenesis in-vivo.

As these processes are anchorage-dependent, the maturing organoids are compressed into a bulk, to provide them with viscous or stiff surrounding material (such as edible mycelium fibers or ECM proteins), to improve fiber polarity and muscle cell fusion. In the final maturation phase of the whole-cut skeletal muscle bulk, perfusable channels are created by the injection of sacrificial material, delaying the formation of necrotic cores. Such differentiation and maturation procedures may be monitored using 3D live imaging, 3D immunostaining, and FISH, to increase efficiency, accuracy and yield.

Thus, according to some embodiments, there is provided a method for integrating large numbers of myogenic organoids into a whole-cut tissue containing long, inter-organoid muscle fibers. To this aim, myofiber maturation in single skeletal muscle organoids is attained by attaching the organoid to various substrates. Further, inter-organoid fusion and co-maturation of pairs of organoids are attained by utilizing conditions and timing for optimal fusion and co-maturation of organoids, to obtain cross-organoid, long muscle fibers, by examining, inter alia, the enhancement of cross-organoid interactions with addition of protein fibers. Additionally, self-assembly of a “pile of organoids” is attained, to form a thick, whole-cut tissue, by providing conditions for the compression of a bulk of organoids (optionally mixed with protein fibers), so as to form a thick and mature skeletal muscle tissue. Optionally, to avoid the formation of necrotic cores in particular in thick bulks of compressed organoids due to lack of oxygen and nutrient availability, the generation of perfusable channels by the injection of thermoreversible sacrificial material may be performed.

According to some embodiments, maturated muscle fibers in a single organoid may be formed by fusion of myogenic progenitor cells (myoblasts, myocytes, and the like) to form muscle fibers (myotubes, myofibrils, and the like) in single organoids, attached to anchoring substrates. Elongated cross-organoid skeletal muscle fibers formed between pairs of fused organoids may be obtained by physically constraining pairs of organoids and by the addition of minimal biochemical manipulations that mimic signals involved in myogenesis, as myocytes from different organoids fuse into cross-organoid myofibers. Thereafter, a thick, high density network of organoids with multiple mature muscle fibers in the length-scale of several organoids are formed. In some embodiments, the optimization of muscle alignment, maturation and hypertrophy may further make use of chemical stimulations such as Myostatin inhibition, or electrical stimulations.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1: Generation of Organoids Comprising Myogenic Progenitor Cells from bESCs

### Cell Lines and Culture

The bovine ESC line CJ-E (Bogliotti et al, 2018, PNAS 115(9): 2090-2095) was used in all experiments. Cells were cultured in 2D plates as described (Soto et al., Scientifc Reports (2021) 11:11045). Briefly, bESCs were cultured in NBFR medium (N2B27 medium supplemented with 1% BSA, 20 ng/mL FGF2 and 2.5 μM IWR1, Soto et al. 2021), further supplemented with activin-A (20 ng/mL) on top of MEF feeder cells on gelatin coated plates. N2B27 medium is a serum-free medium suitable for neural stem cell and pluripotent stem cell proliferation, containing DMEM/High Glucose plus N2 (Thermo Scientific) and B27 (Thermo Scientific).

### Aggregation

Cells in 80% confluent wells were dissociated using TrypLE™ (cat #12604013, ThermoFischer). For aggregation, cells were either seeded at a concentration of 54,000 cells/mL in 24-well plates (0.5 mL per well) and incubated at a shaking speed of 150 rpm, or seeded as single droplets in a low-adherence 96-well U-bottom plate (Greiner) at a concentration of 500-1000 cells per 40 μl droplet and incubated without shaking. In both cases, aggregation was performed in differentiation medium (next section) under 37C° and 5% CO2 conditions.

### Organoid Differentiation

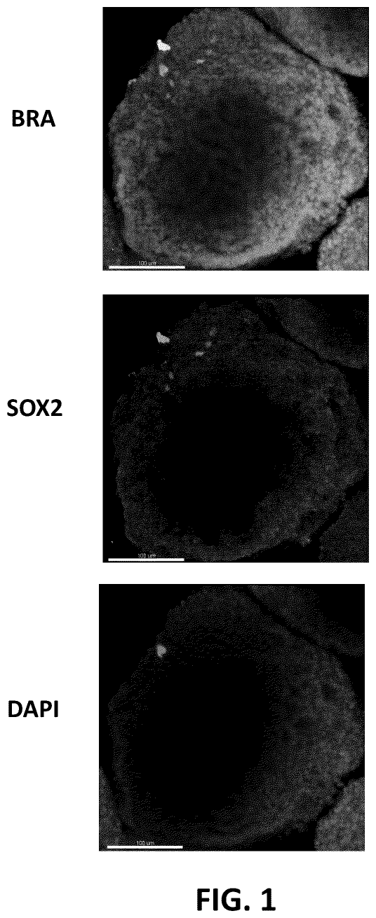

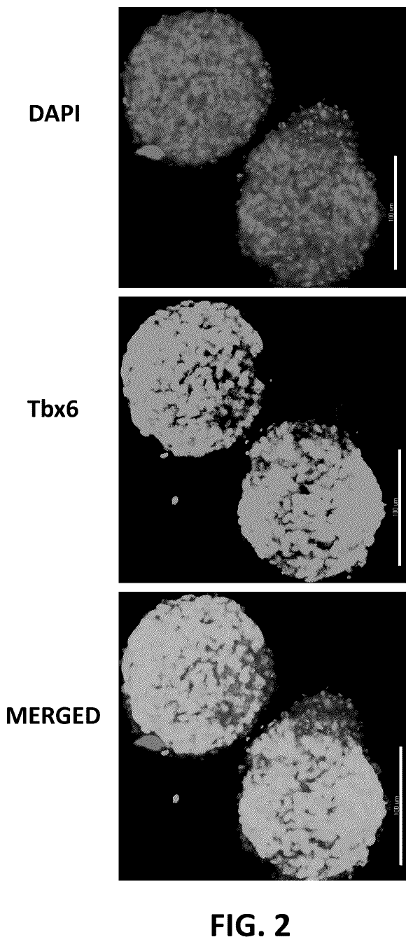

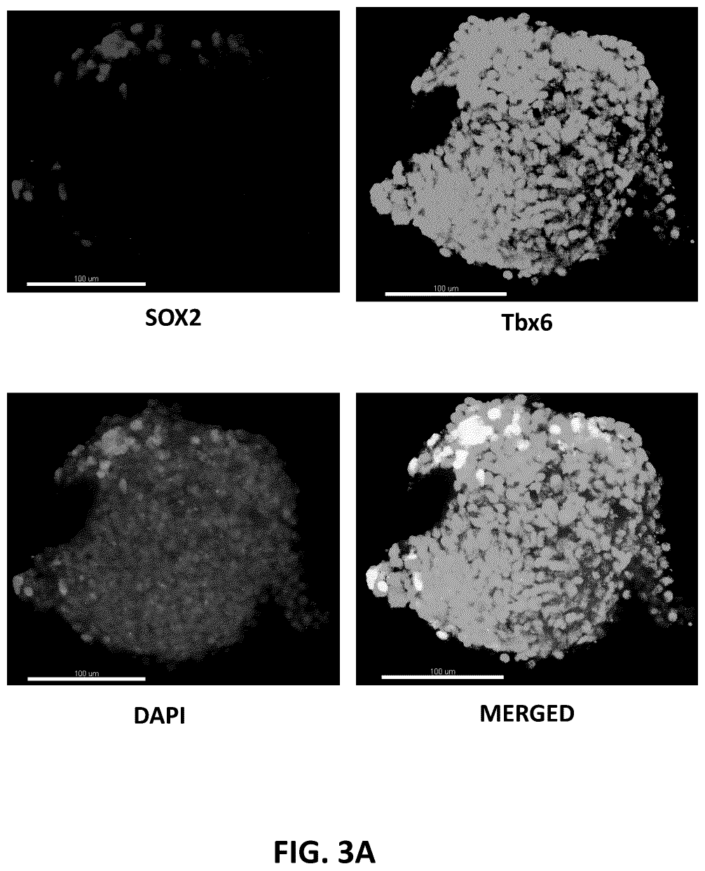

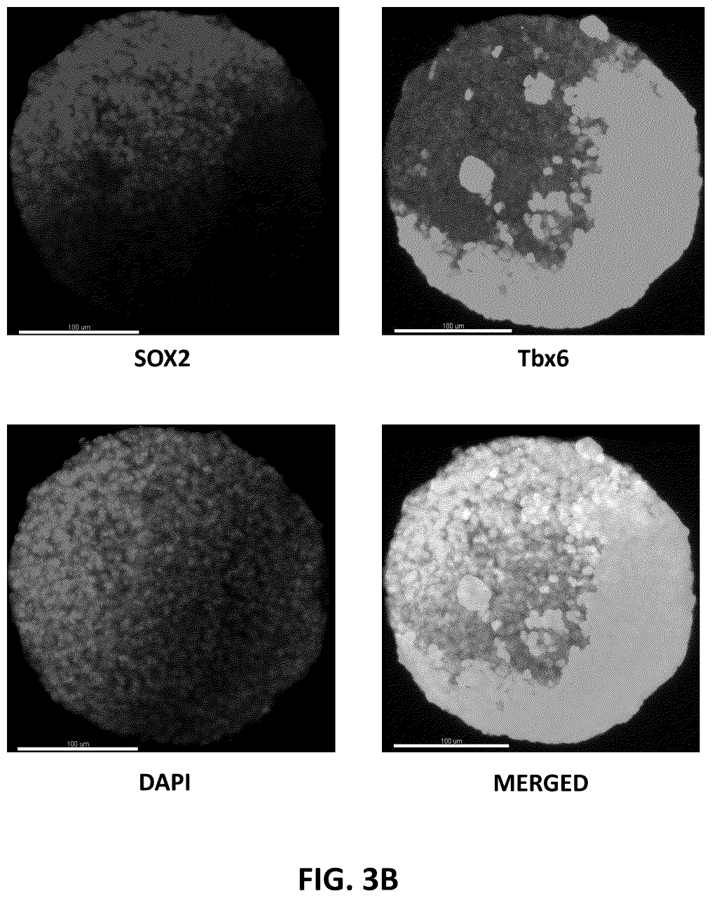

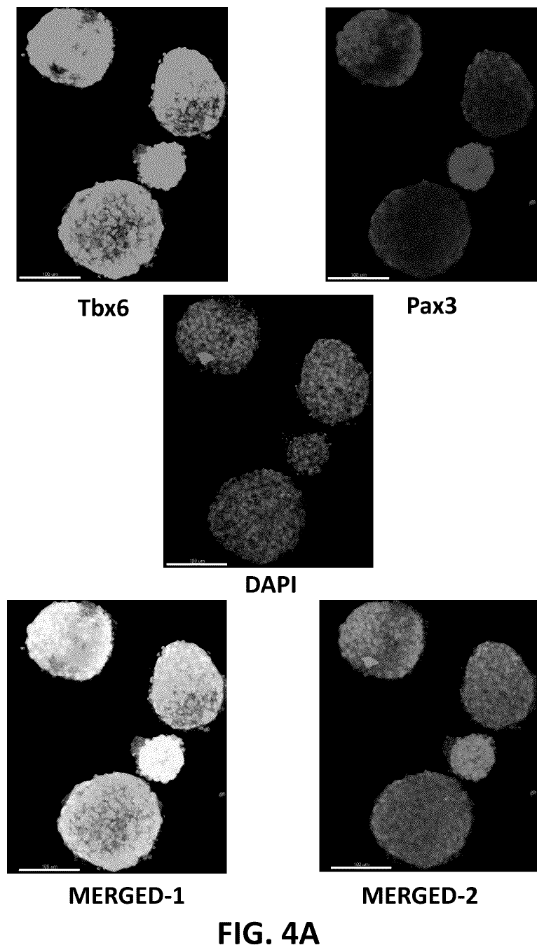

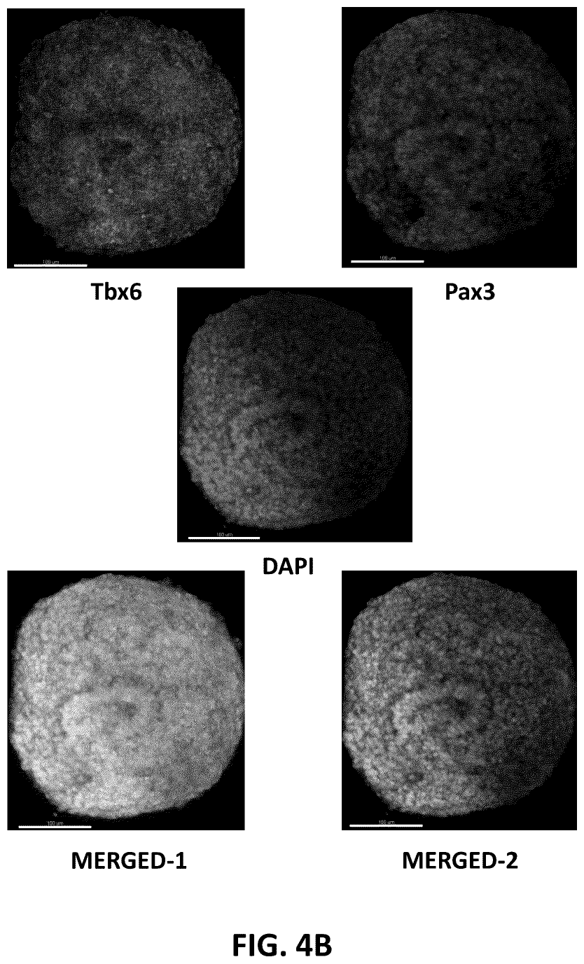

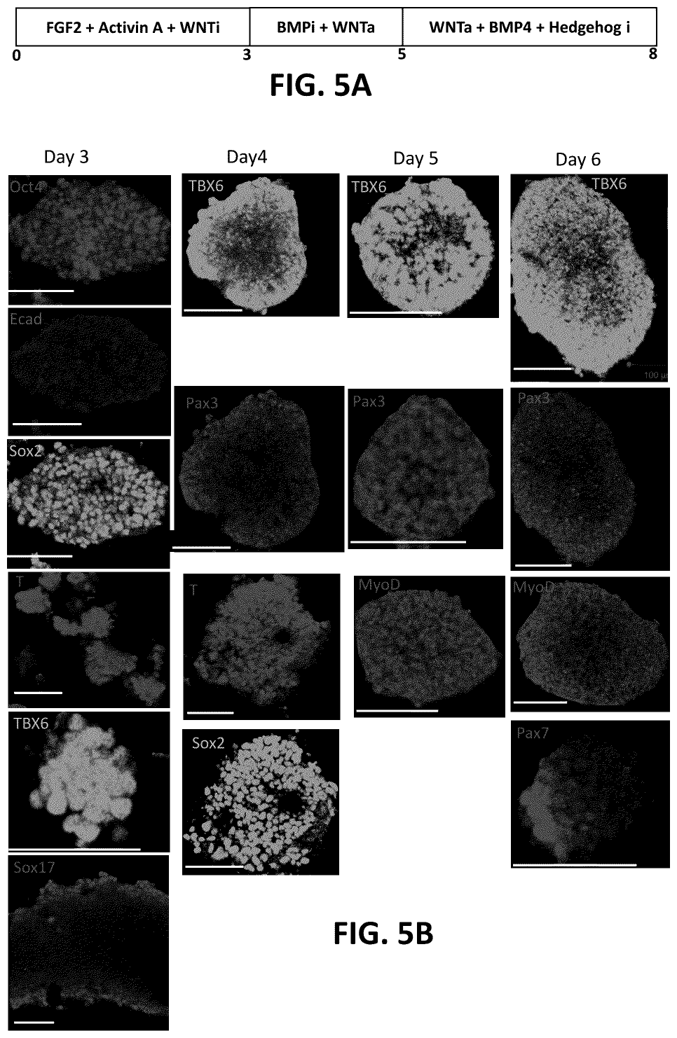

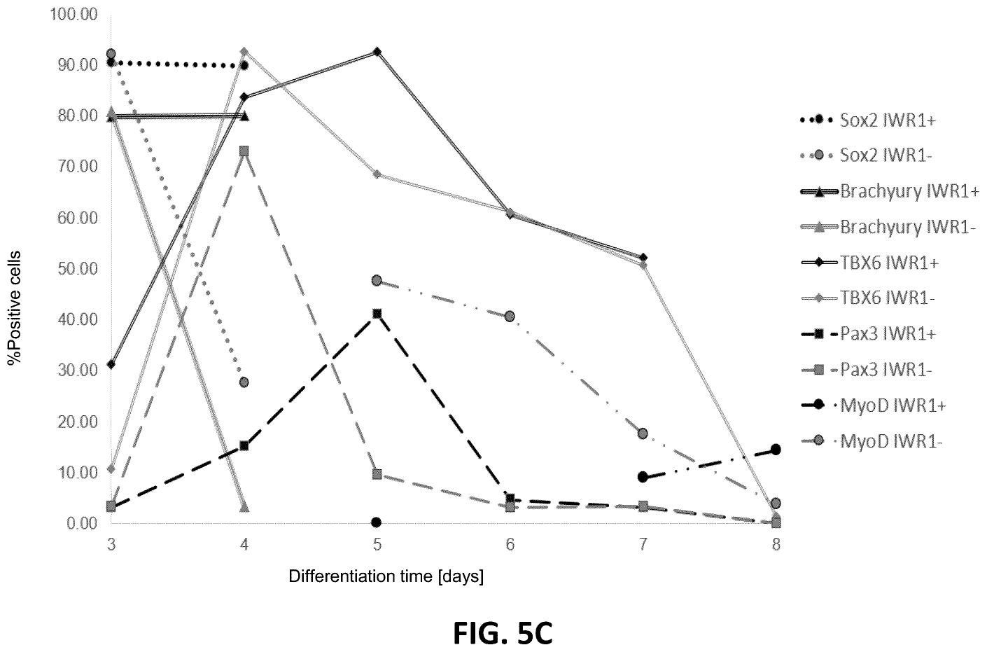

For differentiation, NBFR medium of the preceding step was used, with the following modifications: 1) IWR1(−)/activin A(+); 2) IWR1(−)/activin A(−); and 3) IWR1(+)/activin A(+). After 3 days, the presence of early mesodermal cells was confirmed by immunostaining against Brachyury, (red, AF2085, R&D Systems), as can be seen in FIG. 1. Organoids were then transferred to NBFR without IWR1 and with the addition of CHIR (Chir99021, 10 um, SML1046, Sigma), LDN-193189 (0.5 uM, SML0559, Sigma), and 5% KSR (10828028, Gibco) for 2 days, to obtain posterior paraxial mesoderm, confirmed by immunostaining against Tbx6 (green, ab38883, Abcam), as can be seen in FIGS. 2, 3. After 1 more day, anterior paraxial mesoderm was obtained, confirmed by immunostaining against Pax3 (red, AB_528426, DSHB). At this stage, somite-like structures, visible as spherical cell structures exhibiting closed-loop shapes of Pax3+ cells) may (FIG. 4B) or may not (FIG. 4A) be observed. Organoids were then transferred to N2B27 supplemented with CHIR (3 uM)+GDC-0449 (150 nM, Selleck)+Bmp4 (50 ng/mL, R&D Systems) for 1 day, to obtain myogenic progenitor cells, confirmed by immunostaining against MyoD (red, PCRP-MYOD1-2A5-s, DSHB) and Pax7 (red, PAX7-s, DSHB), as can be seen in FIG. 5B. FIG. 5C presents the effect of the addition of the Wnt inhibitor IRW1 at step (a), days 0-3, on expression of the tested markers. As can be seen, Sox2 was expressed on the cells on day 3 in both cases, but while the expression level was maintained on day 4 with IRW1 present, it was much reduced on day 4 in the absence of IRW1; Brachyury expression showed the same pattern as Sox2; Tbx6 expression went up in both cases from day 3 to day 4, and with IRW1 it kept increasing to a peak on day 5 with followed by declining until day 7, while without IRW1 Tbx6 expression was decreased on day 5 but from day 6 it was similar to the level with IRW1; Pax3 expression with IRW1 increased from day 3 to day 5, then decreased, while without IRW1 it sharply increased to day 4 and then sharply decreased till day 5, and then was the same as with IRW1 from day 6 on; MyoD appeared on day 7 with IRW1 and slightly increased on day 8, while without IRW1 it appeared already on day 5, and then declined till day 8.

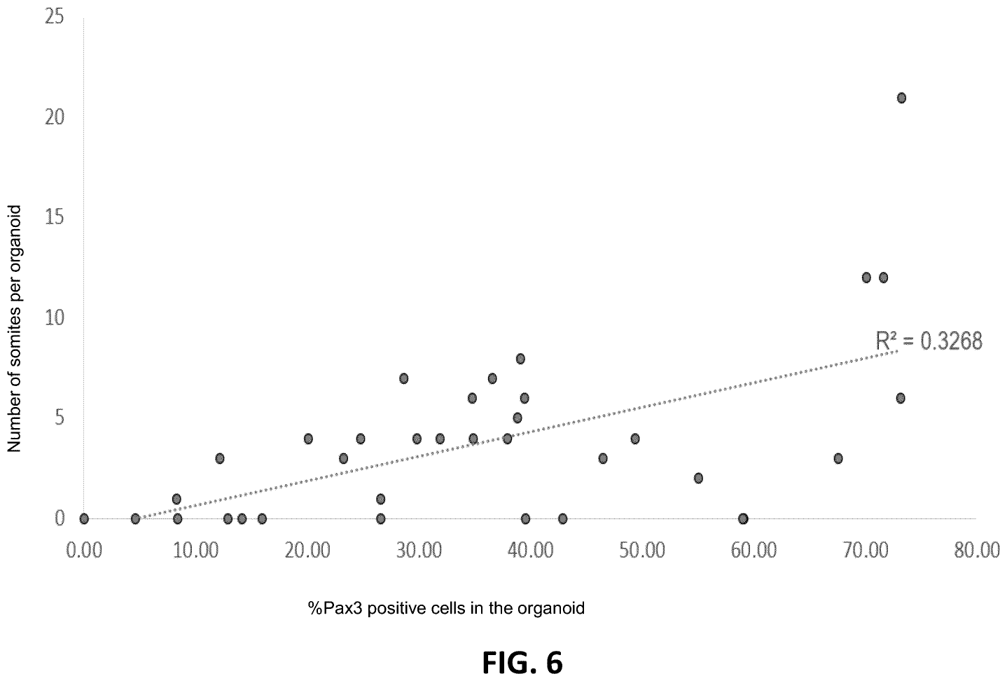

The positive correlation between the number of Pax3+ cells and the number of somites shown in FIG. 6 suggests that most Pax3-positive cells in the organoids appear in somites, rather than in other non-myogenic (e.g. neural) structures.

Altogether, the results presented above demonstrate the successful formation of myogenic progenitors in a culture staring with bESC, in an efficient, timely, and cost-effective manner.

### Example 2: Generation of Somites and Myogenic Progenitor Cells from bESCs

The 3D differentiation model disclosed herein forms basis for industry-level scaleup processes and adaptation to other edible species. A full suspension, cell-cluster based protocol as disclosed herein can be incorporated in bioreactors of unlimited size. Moreover, the protocol is serum free, and does not involve genetic modifications, rendering it suitable for industrial processing.

The following exemplary protocol provides bovine somites from bovine ES cells. The protocol includes the following steps:

Step a—bESC Aggregate Formation:

Aggregate spheres of about 50-300 bESCs were obtained by seeding the bESCs in a first medium (N2B27 or NDIFF® medium, e.g. at an ULA u-bottom plate, or multi-well plates). The first medium may be supplemented with Wnt inhibitor (for example, IWR1 at a concentration of about 1-10 mM). The first medium may optionally include one or more growth factors and/or other agents, such as, activin A. Optionally, a Rock inhibitor may also be added. Aggregate spheres of bESCs may also be obtained by culturing the bESCs at a density of about 5K-100K cells/ml in a similar medium. The cells may be grown under shaking/agitation conditions.

At the end of the first step (a), early mesodermal progenitors are visible and constitute most of the cells comprising the aggregates.

Step b—Paraxial Mesoderm Formation:

The first medium is replaced with a second, fresh medium (for example, N2B27), which does not include the supplements used in step (a). After a period of time of between 0-72 hours, at least one Wnt activator (for example, as Chir99201 or Rspo3 at a concentration in the range of about 1-10 mM) and/or at least one BMP inhibitor (such as LDN-193189 or Noggin at a concentration of about 0.1-5 mM) may be added. Optionally, 1-10% KSR may be added to the medium.

After an incubation period of about 10-96 hours in the presence of one or more of the Wnt activator, BMP inhibitor and/or KSR, the medium may optionally be replaced, or the agents be removed from the medium.

Early paraxial mesoderm progenitors are visible about 1-2 days after early mesodermal progenitors are generated.

Step c—Somite Formation:

About 1-2 days after the formation of the early paraxial mesoderm progenitors, matured paraxial mesoderm progenitors (PSM) and somite-like structures are visible.

### Example 3: Differentiation of Organoids Including Somite-Like Structures to Myogenic Progenitors

The following exemplary protocol provides myogenic progenitors from the generated bovine somites. The protocol includes the following steps:

1 day after the formation of the somite-like structures, the medium is changed to a third medium, (for example, to N2B27) supplemented with one or more of: Wnt activator (such as 1-10 mM Chir99201 or Rspo3); Hedgehog inhibitor (such as 50-300 nM GDC0449, GANT58, or GANT-61); 1-15% KSR; a growth factor (such as, 2-50 ng/ml HGF, 0.5-10 ng/ml IGF, 2-100 ng/ml FGF2. 0.5-5% Insulin), extra cellular matrix (ECM) (such as 1-20% Matrigel or ECM components such as fibronectin, collagen, or laminin), micro-scaffolds or protein fibers/filaments (such the SpheroSeev and mycelium).

Two days after the medium change, myogenic progenitors, such as myoblasts, are visible by staining the organoids with myogenic markers, such as MyoD or Pax7. Pax3-positive and MyoD-negative cells represent brown and/or white adipogenic progenitors.

### Example 4: Differentiation of Myogenic Progenitors to Myocytes and Muscle Fibers

Starting from myogenic progenitors, the medium is changed to a fourth medium (for example, N2B27), which is devoid of at least Hedgehog inhibitor, compared to the previous, third, medium. The fourth medium may optionally be supplemented with the other supplements added to the third medium (Wnt activator, KSR; a growth factor, ECM, and/or micro-scaffolds or protein fibers/filaments).

1-4 days after addition of the fourth medium, myocytes are visible.

1. A method for generating bovine myogenic progenitor cells, the method comprising:
(a) culturing bovine pluripotent stem cells (bPSCs) in a first culture medium comprising at least one growth factor for a first time period until aggregates form and the cells express Brachyury, thereby obtaining mesodermal organoids;
(b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and optionally somite-like structures;
(c) replacing the second culture medium with a third culture medium comprising at least one Wnt activator and at least one hedgehog inhibitor and incubating for a third time period until the cells express MyoD, thereby obtaining myogenic progenitor cells.

2. The method of claim 1, wherein the bPSCs are bovine embryonic stem cells (bESCs).

3. The method of claim 1, wherein the at least one growth factor is selected from the group consisting of a fibroblast growth factor (FGF), an epidermal growth factor (EGF), a hepatocyte growth factor (HGF), an insulin-like growth factor (IGF), a leukemia inhibitory factor (LIF), Insulin, and Transferrin.

4. (canceled)

5. The method of claim 1, wherein the at least one growth factor is FGF2.

6. The method of claim 1, wherein the concentration of the at least one growth factor in the first culture medium is from about 1 to about 100 ng/mL.

7. The method of claim 1, wherein the first culture medium further comprises at least one Wnt inhibitor and/or activin A.

8. The method of claim 7, wherein the at least one Wnt inhibitor is selected from the group consisting of IWR1, XAV939, IWP-2, IWP-3, IWP-4, and iCRT3.

9. The method of claim 7, wherein the at least one Wnt inhibitor is IWR1.

10. (canceled)

11. (canceled)

12. (canceled)

13. The method of claim 1, wherein the culturing or incubating at any of steps (a)-(c) is done in suspension.

14. (canceled)

15. The method of claim 1, wherein the at least one BMP inhibitor is selected from the group consisting of LDN-193189, K02288, sclerostin, chordin, noggin, CTGF, follistatin, gremlin, inhibin, and BMP-3.

16. (canceled)

17. The method of claim 1, wherein the at least one Wnt activator is selected from the group consisting of Chir99021, Wnt3a, and Rspo3.

18. (canceled)

19. The method of claim 1, wherein the length of the first time period is from about 48 hours to about 96 hours; the length of the second time period is from about 24 hours to about 96 hours; and/or the length of the third time period is from about 10 hours to about 48 hours.

20. The method of claim 1, wherein step (b) further includes in intermediate step (b1), comprising replacing the second culture medium, after the cells have differentiated into posterior paraxial mesodermal organoids, with an intermediate culture medium of the same composition, or with an intermediate culture medium comprising a higher concentration of the at least one Wnt activator.

21. (canceled)

22. The method of claim 1, wherein the at least one hedgehog inhibitor is selected from the group consisting of GDC-0449, GANT58, and GANT-61.

23. (canceled)

24. The method of claim 1, wherein the third culture medium further comprises one or more of Knock-out™ Serum Replacement (KSR), a growth factor, a BMP pathway activator, and an extracellular matrix (ECM) material.

25. (canceled)

26. (canceled)

27. (canceled)

28. The method of claim 1, wherein the bPSCs express at least one of Oct4 and Sox2: the mesodermal organoids further express Sox2; and/or the myogenic progenitor cells further express Pax7.

29. (canceled)

30. (canceled)

31. The method of claim 1, wherein the bPSCs are cultured in step (a) at a density of about 5,000-100,000 cells/mL; and/or each of the mesodermal organoids obtained in step (a) comprises, on average, about 50-300 bPSCs.

32. (canceled)

33. A method for producing bovine somite-like structures, the method comprising:
(a) culturing bovine pluripotent stem cells (bPSCs) in a first culture medium comprising at least one growth factor for a first time period until aggregates form and the cells express Brachyury, thereby obtaining mesodermal organoids; and
(b) replacing the first culture medium with a second culture medium comprising at least one bone morphogenic protein (BMP) inhibitor and at least one Wnt activator and essentially devoid of a Wnt inhibitor and activin A, and incubating for a second time period until the cells express Tbx6 and Pax3, thereby obtaining anterior paraxial mesodermal organoids and somite-like structures.

34. The method of claim 33, further comprising:
(c) replacing the second culture medium with a fresh culture medium comprising at least one Wnt activator and at least one hedgehog inhibitor and incubating until the cells express MyoD, thereby obtaining myogenic progenitor cells.

35. (canceled)

36. (canceled)

37. A cultured meat product comprising an organoid comprising myogenic progenitor cells obtained by the method of claim 1.
